(19) United States
(12) Patent Application Publication
(10) Pub. No.: US 2025/0208111 A1
(43) Pub. Date: Jun. 26, 2025

(54) WATER ANALYZER WITH INDICATOR

(71) Applicant: ZODIAC POOL CARE EUROPE, Belberaud (FR)

(72) Inventor: Simon Duffaut, Castelginest (FR)

(73) Assignee: ZODIAC POOL CARE EUROPE, Belberaud (FR)

(21) Appl. No.: 18/989,291

(22) Filed: Dec. 20, 2024

Related U.S. Application Data
(60) Provisional application No. 63/614,501, filed on Dec. 22, 2023.

(51) Int. Cl. G01N 33/18; E04H 4/14

(52) U.S. Cl. CPC G01N 33/1886; E04H 4/14

(57) ABSTRACT

A water analyzer for a swimming pool or spa includes an indicator, which may be utilized for providing information to a user. The information provided to the user may include, but is not limited to, water characteristic information, environmental information, and/or information about the water analyzer. A method of analyzing water of a swimming pool or spa may include providing a water analyzer within the swimming pool or spa and controlling an indicator on the water analyzer to provide information to a user.

### REFERENCE TO RELATED APPLICATION

This application claims the benefit of and priority to U.S. Provisional Patent Application No. 63/614,501, filed on Dec. 22, 2023, and entitled WATER ANALYZER WITH INDICATOR, the content of which is hereby incorporated by reference in its entirety.

### FIELD OF THE INVENTION

The invention relates to systems and apparatuses for fluid analyzers, such as water analyzers for swimming pools and spas.

### BACKGROUND OF THE INVENTION

Water analyzers may be utilized with water-containing vessels such as swimming pools or spas (hereinafter “swimming pools”) to monitor or detect characteristics of water of the swimming pools such as water temperature, oxidative reduction potential (ORP), pH, salinity, water level, etc. Water analyzers may float within the swimming pool, but the data obtained by the water analyzers have traditionally only been accessible using an external device, such as an application running on a user's smartphone. As such, unless a user has such an external device, the user is unable to obtain information from the water analyzer.

### SUMMARY

Embodiments covered by this patent are defined by the claims below, not this summary. This summary is a high-level overview of various embodiments and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to some embodiments, a water analyzer for a swimming pool or spa, the water analyzer includes an indicator for providing information to a user.

According to various embodiments, a method of analyzing water of a swimming pool or spa includes providing a water analyzer within the swimming pool or spa and controlling an indicator on the water analyzer to provide information to a user.

Various implementations described herein can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The specification makes reference to the following appended figures, in which use of like reference numerals in different figures is intended to illustrate like or analogous components.

FIG. 1 illustrates a water analyzer for a pool system according to embodiments.

FIG. 2 illustrates a pool system with the water analyzer of FIG. 1

### DESCRIPTION OF THE INVENTION

Described herein are systems and methods for providing information obtained by a water analyzer locally on the water analyzer. In certain embodiments, the systems and methods described herein may allow a user to obtain information about one or more characteristics of the water of the swimming pool or spa without requiring an external device. In some embodiments, the systems and methods described herein may provide information related to water treatment balancing. In various embodiments, the systems and methods described herein may utilize an indicator to provide information to the user. The indicator may be various devices or features as desired, such as but not limited to one or more light sources, a screen or display, a color roll, electronic ink, other devices and/or features that may or may not require energy consumption to provide information to the user, combinations thereof, and/or other indicators as desired. In certain embodiments, the water analyzer with the indicator may allow for the water analyzer to display the information to a user even when the pool cover is deployed on the pool surface

In one non-limiting example, the indicator includes one or more light sources, such as but not limited to one or more light emitting diodes (LEDs), and one or more characteristics of light emitted from the one or more light sources may be controlled to provide information to the user. As non-limiting examples, a color, brightness, pattern of light, combinations thereof, and/or other characteristics of the emitted light may be controlled. In one non-limiting example, the color of the light may be controlled to provide information to a user, such as a first color indicating that all parameters are within an acceptable level or range, a second color indicating that one parameter is outside of an acceptable range but the water is safe to use, and a third color indicating that at least one parameter is outside of an acceptable range and the water is not safe.

The information provided locally on the water analyzer may be provided in various intervals as desired. As non-limiting examples, the information may always be displayed, displayed at certain times of days, displayed responsive to a trigger event, displayed for a predetermined amount of time, displayed in response to a prompt or inquiry from a user, and/or as otherwise desired.

In some embodiments, the indicator providing the information may be on a portion of the water analyzer above the waterline of the pool when the water analyzer is within the pool.

Various other benefits and advantages may be realized with the systems and methods provided herein, and the aforementioned advantages should not be considered limiting.

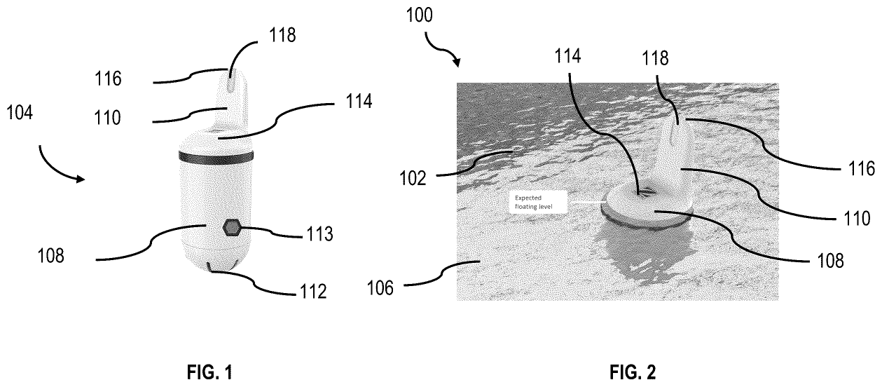

FIGS. 1 and 2 illustrate a pool system 100 according to embodiments. The pool system 100 generally includes a pool 102 and a water analyzer 104.

The pool 102 may have various features, arrangements, shapes, and profiles as desired.

The water analyzer 104 may be provided within water 106 of the pool 102. In certain embodiments, the water analyzer 104 includes one or more probes or sensors 113. While a single sensor 113 is illustrated, in other embodiments, any number of sensors 113 may be included as desired. The one or more sensors 113 may be provided at various locations on and/or within the water analyzer 104 as desired, and the location illustrated in FIG. 1 should not be considered limiting.

In some embodiments, the one or more probes or sensors 113 may be provided for detecting or measuring one or more characteristics of the water 106. As non-limiting examples, the one or more water characteristics may include, but are not limited to, oxidation-reduction potential (ORP), pH, temperature, conductivity, salinity, water level, other types of water characteristics, and/or combinations thereof. While a single sensors 113 is illustrated, in other embodiments, the water analyzer 104 may include a plurality of sensors 113 for detecting a plurality of water characteristics.

Additionally, or alternatively, one or more sensors 113 of the water analyzer 104 may include one or more sensors for detecting or measuring a characteristic of the water analyzer 104 and/or environmental data. As non-limiting examples, the one or more sensors 113 may detect environmental data as additional information sensed or detected, such as but not limited to, air temperature, wind force, wave force may include air temperature, wind force, wave force, humidity, day/night (e.g., time of day), combinations thereof, and/or as otherwise desired. As further non-limiting examples, the one or more sensors 113 may detect information abou the water analyzer 103, such as but not limited to battery capacity, battery or charging status, communication status, combinations thereof, and/or other information.

As best illustrated in FIG. 1, the water analyzer 104 generally includes a submergible portion 108 and a projecting portion 110. The water analyzer 104 may include devices, characteristics or mechanisms such that the water analyzer 104 is buoyant and may float within the pool 102. The submergible portion 108 includes a bottom end 112, which may be the bottom end of the water analyzer 104, and a top end 114. In certain embodiments, and as illustrated in FIG. 2, at least the bottom end 112 is submerged within the water 106 when the water analyzer 104 is provided within the pool 102. Optionally, the top end 114 may be submerged, although it need not be in other embodiments.

As illustrated in FIGS. 1 and 2, the projecting portion 110 of the water analyzer 104 extends upwards from the top end 114 of the submergible portion 108. In certain embodiments, the projecting portion 110 has a height such that the projecting portion extends above a waterline of the pool 102 when the water analyzer 104 is provided within the pool 102. The projecting portion 110 includes a top end 116, which may be the top end of the water analyzer 104. In certain embodiments, the projecting portion 110 optionally is offset relative to a center axis of the submergible portion 108. In such embodiments, the offset nature of the projecting portion 110 may facilitate providing information to a user as described in detail below. Optionally, the offset projecting portion 110 may passively promote movement of the water analyzer 104 within the pool 102 (e.g., by creating an unbalanced weight distribution above the water, optionally allowing for wind, waves, water flow, etc. to push or otherwise cause movement of the water analyzer 104).

As illustrated in FIGS. 1 and 2, the water analyzer 104 includes one or more indicators 118 for providing information to a user. As discussed in detail below, the information provided to the user may be information about one or more water characteristics (e.g., as obtained by the one or more sensors of the water analyzer 104), information about components of the water analyzer 104 (e.g., battery status, communication status), and/or environmental information (e.g., wind force, air temperature, wave force, etc.).

In certain embodiments, the one or more indicators 118 may be provided on a portion of the water analyzer 104 configured to be above the waterline of the pool 102 when the water analyzer 104 is provided within the pool 102. As non-limiting examples, the one or more indicators 118 may be provided on the projecting portion 110, optionally proximate to and/or at the top end 116 of the projecting portion 110. Additionally, or alternatively, the one or more indicators 118 may be provided proximate to and/or at the top end 114 of the submergible portion 108. In FIG. 1, the indicator 118 is provided on the projecting portion 110. In other embodiments, the indicator 118 may be provided on a portion of the water analyzer 104 that is submerged within the pool 102.

The indicator 118 may be various devices, mechanisms, or features for providing information to a user locally on the water analyzer 104. As non-limiting examples, the indicator 118 may be a screen, display, one or more light sources, a color roll, electronic ink, other devices and/or features that may or may not require energy consumption to provide information to the user, combinations thereof, and/or other devices. In the embodiment illustrated in FIG. 1, the indicator 118 includes a display and one or more light sources, such as one or more LEDs. In other embodiments, the indicator 118 includes only a display, only a light source, combinations thereof, and/or other types of displays or combinations thereof as desired.

In one non-limiting example, the indicator 118 may be a device and/or feature that does not require energy consumption to provide and/or display the information to the user, such as but not limited to the color roll and/or electronic ink. In such examples, energy may be used for changing information but is not required for the display. As an example, a color roll may be a device (such as but not limited to a cylinder, rolling blades, etc.) with a plurality of different colors, symbols, numbers, markings, and/or other information, on it, and a masking device may mask certain information and/or only allow for certain information to be visible (e.g., a particular color). In this example, energy may be utilized to change what is masked or unmasked, but no energy is required to display the information. As another non-limiting example, electronic ink may be utilized as the indicator, and while energy may be utilized to change the information presented, no energy is required to display the information. Various other devices and/or features that may or may not require energy consumption to provide information to the user may be utilized as desired.

In certain embodiments, one or more characteristics of the indicator 118 may be controlled to provide information to the user. In the example where the indicator 118 includes one or more light sources, one or more characteristics of light emitted from the one or more light sources may be controlled to provide various information to the user. As non-limiting examples, a color of the emitted light, brightness of the emitted light, pattern of the emitted light, status of the emitted light (e.g., on or off), combinations thereof, and/or other characteristics may be controlled.

As one non-limiting example, the indicator 118 may emit light with a first color (e.g., green) to indicate that one or more parameters (e.g., water characteristic data, environmental data, data about the water analyzer, etc.) are all operating and/or within acceptable tolerances or ranges. The indicator 118 may emit a second color (e.g., orange) to indicate that one or more parameters are outside of the acceptable tolerance or range, but the water 106 of the pool 102 is safe to use. As a non-limiting example, the second color may indicate that battery capacity of the water analyzer is low, but the water 106 of the pool 102 is safe to use. In some embodiments, the water analyzer 104 (and/or a controller such as a process/memory of the water analyzer 104) may determine the water 106 is safe to use based on the type of parameter and/or the parameter information relative to the threshold or range. The indicator 118 may emit a third color (e.g., red) to indicate that one or more parameters are outside of the acceptable tolerance or range, and the water 106 of the pool 102 is not safe to use. As a non-limiting example, the third color may indicate that the pH levels are outside of the acceptable tolerance or range and thus the pool 102 is not safe to use. Other information or combinations of information may be conveyed and by controlling various characteristics of the indicator 118.

In certain embodiments, the indicator 118 may be activated and/or provide the information at various time intervals and/or for various durations as desired.

In some embodiments, the indicator 118 may continuously provide the information (e.g., the indicator 118 may be always on).

Additionally, or alternatively, the indicator 118 may provide the information at certain times of day and/or pursuant to a schedule.

Additionally, or alternatively, the indicator 118 may provide the information responsive to a trigger event. As non-limiting examples, the indicator 118 may provide information responsive to a user action such as but not limited to flipping the water analyzer 104, tilting the water analyzer 104, rotating the water analyzer 104, tapping the water analyzer 104, combinations thereof, and/or as otherwise desired. As a non-limiting example, responsive to a user flipping the water analyzer 104 within the pool 102, the water analyzer 104 may convey, via the indicator 118, information about a water characteristic, information about an environmental characteristic, and/or information about the water analyzer 104 itself. In one non-limiting example, responsive to the user flipping the water analyzer 104, the water analyzer 104 may provide an indication of ORP, pH, water temperature, and/or salinity. In this regard, the user may not need a smartphone or other device to obtain information from the water analyzer 104, and/or may facilitate a user within the pool 102 obtaining real-time information from the water analyzer 104.

In certain embodiments, the water analyzer with the indicator 118 may allow for the water analyzer to display the information to a user even when the pool cover is deployed on the pool surface. As a non-limiting example, the indicator 118 provided on the offset projecting portion 110 may allow for the water analyzer 104 to accommodate a deployed pool cover and still project the indicator 118 above the waterline and/or above the pool cover.

Exemplary concepts or combinations of features of the invention may include:
A. A water analyzer for a swimming pool or spa, the water analyzer comprising an indicator for providing information to a user.
B. The water analyzer of any preceding or subsequent statement or combination of statements, wherein the information comprises at least one of water characteristic information, environmental information, or information about the water analyzer.
C. The water analyzer of any preceding or subsequent statement or combination of statements, wherein the indicator is a display or screen.
D. The water analyzer of any preceding or subsequent statement or combination of statements, wherein the indicator includes one or more light sources.
E. The water analyzer of any preceding or subsequent statement or combination of statements, wherein the indicator is on a portion of the water analyzer above a waterline of the swimming pool or spa when the water analyzer is provided within the swimming pool or spa.
F. The water analyzer of any preceding or subsequent statement or combination of statements, wherein the water analyzer comprises a submergible portion and a projecting portion, and wherein the indicator is on a top end of the submergible portion or the projecting portion.
G. The water analyzer of any preceding or subsequent statement or combination of statements, wherein the indicator comprises a light source, and wherein at least one characteristic of light emitted from the light source is controlled to provide the information to the user.
H. The water analyzer of any preceding or subsequent statement or combination of statements, wherein the at least one characteristic comprises a brightness of light, color of light, pattern of light, and/or status of light.
I. The water analyzer of any preceding or subsequent statement or combination of statements, wherein the indicator provides:
   a. a first indication based on a measured or detected parameter being within a predefined range or tolerance and the water being safe to use;
   b. a second indication based on the measured or detected parameter being outside the predefined range or tolerance and the water being safe to use; and
   c. a third indication based on the measured or detected parameter being outside the predefined range or tolerance and the water being unsafe to use.
J. The water analyzer of any preceding or subsequent statement or combination of statements, wherein the indicator displays information without consuming energy.
K. The water analyzer of any preceding or subsequent statement or combination of statements, wherein the indicator comprises a color roll and/or electronic ink.
L. The water analyzer of any preceding or subsequent statement or combination of statements, wherein the water analyzer is configured to display the information to a user using the indicator even when a pool cover is deployed on a pool surface.
M. The water analyzer of any preceding or subsequent statement or combination of statements, wherein the information comprises water characteristic information, optionally wherein the water characteristic information comprises at least one of ORP, pH, water temperature, waterline level, or salt concentration.
N. The water analyzer of any preceding or subsequent statement or combination of statements, wherein the information comprises environmental information, optionally wherein the environmental information comprises at least one of wave force, wind force, or air temperature.
O. The water analyzer of any preceding or subsequent statement or combination of statements, wherein the information comprises information about the water analyzer, optionally wherein the information about the water analyzer comprises a battery capacity, a communication status, or a battery status.
P. The water analyzer of any preceding or subsequent statement or combination of statements, wherein the water analyzer is configured to provide information responsive to a trigger event.
Q. The water analyzer of any preceding or subsequent statement or combination of statements, wherein the trigger event comprises at least one of flipping the water analyzer, tilting the water analyzer, rotating the water analyzer, or tapping the water analyzer.
R. The water analyzer of any preceding or subsequent statement or combination of statements, wherein the water analyzer is configured to provide information at various time intervals and/or for various durations as desired.
S. The water analyzer of any preceding or subsequent statement or combination of statements, wherein the water analyzer is configured to provide information continuously.
T. The water analyzer of any preceding or subsequent statement or combination of statements, wherein the water analyzer is configured to provide information at regular or irregular intervals of time.
U. The water analyzer of any preceding or subsequent statement or combination of statements, wherein the water analyzer is configured to provide information to a user without requiring a smartphone or user device.
V. A pool system comprising the water analyzer of any preceding or subsequent statement or combination of statements.
W. A method of analyzing water of a swimming pool or spa, the method comprising:
   a. providing a water analyzer within the swimming pool or spa; and
   b. controlling an indicator on the water analyzer to provide information to a user.
X. The method of any preceding or subsequent statement or combination of statements, wherein the information comprises at least one of water characteristic information, environmental information, or information about the water analyzer.

These examples are not intended to be mutually exclusive, exhaustive, or restrictive in any way, and the invention is not limited to these example embodiments but rather encompasses all possible modifications and variations within the scope of any claims ultimately drafted and issued in connection with the invention (and their equivalents). For avoidance of doubt, any combination of features not physically impossible or expressly identified as non-combinable herein may be within the scope of the invention. Further, although applicant has described devices and techniques for use principally with swimming pools and spas, persons skilled in the relevant field will recognize that the present invention conceivably could be employed in connection with other objects and in other manners. Finally, references to “pools” and “swimming pools” herein may also refer to spas or other water containing vessels used for recreation, training, or therapy.

That which is claimed:

1. A water analyzer for a swimming pool or spa, the water analyzer comprising an indicator for providing information to a user.

2. The water analyzer of claim 1, wherein the information comprises at least one of water characteristic information, environmental information, or information about the water analyzer.

3. The water analyzer of claim 2, wherein the information comprises water characteristic information, and wherein the water characteristic information comprises at least one of ORP, pH, water temperature, waterline level, or salt concentration.

4. The water analyzer of claim 2, wherein the information comprises environmental information, and wherein the environmental information comprises at least one of wave force, wind force, or air temperature.

5. The water analyzer of claim 2, wherein the information comprises information about the water analyzer, and wherein the information about the water analyzer comprises a battery capacity, a communication status, or a battery status.

6. The water analyzer of claim 1, wherein the water analyzer is configured to provide the information to the user using the indicator responsive to a trigger event.

7. The water analyzer of claim 6, wherein the trigger event comprises at least one of at least one of flipping the water analyzer, tilting the water analyzer, rotating the water analyzer, or tapping the water analyzer.

8. The water analyzer of claim 1, wherein the information comprises water characteristic information, environmental information, and information about the water analyzer.

9. The water analyzer of claim 1, wherein the indicator is a display or screen.

10. The water analyzer of claim 1, wherein the indicator includes one or more light sources.

11. The water analyzer of claim 1, wherein the indicator is on a portion of the water analyzer above a waterline of the swimming pool or spa when the water analyzer is provided within the swimming pool or spa.

12. The water analyzer of claim 1, wherein the water analyzer comprises a submergible portion and a projecting portion, and wherein the indicator is on a top end of the submergible portion or the projecting portion.

13. The water analyzer of claim 1, wherein the indicator comprises a light source, and wherein at least one characteristic of light emitted from the light source is controlled to provide the information to the user.

14. The water analyzer of claim 13, wherein the at least one characteristic comprises a brightness of light, color of light, pattern of light, and/or status of light.

15. The water analyzer of claim 1, wherein the indicator provides:
a first indication based on a measured or detected parameter being within a predefined range or tolerance and the water being safe to use;
a second indication based on the measured or detected parameter being outside the predefined range or tolerance and the water being safe to use; and
a third indication based on the measured or detected parameter being outside the predefined range or tolerance and the water being unsafe to use.

16. The water analyzer of claim 1, wherein the indicator displays information without consuming energy.

17. The water analyzer of claim 1, wherein the indicator comprises a color roll and/or electronic ink.

18. The water analyzer of claim 1, wherein the water analyzer is configured to display the information to a user using the indicator even when a pool cover is deployed on a pool surface.

19. A method of analyzing water of a swimming pool or spa, the method comprising:
providing a water analyzer within the swimming pool or spa; and
controlling an indicator on the water analyzer to provide information to a user.

20. The method of claim 19, wherein the information comprises at least one of water characteristic information, environmental information, or information about the water analyzer.
